(19) United States
(12) Patent Application Publication
(10) Pub. No.: US 2023/0404639 A1
(43) Pub. Date: Dec. 21, 2023

(54) MEDICAL EXPANSION SYSTEM FOR EXPANDING A LONG BONE, AND KIT

(71) Applicant: OT MEDIZINTECHNIK GMBH, München (DE)

(72) Inventor: Ulrich SCHREIBER, München (DE)

(21) Appl. No.: 18/251,387

(22) PCT Filed: Oct. 29, 2021
(86) PCT No.: PCT/EP2021/080203

§ 371 Date: May 1, 2023

(30) Foreign Application Priority Data
Oct. 31, 2020 (DE) 10 2020 128 704.6

(51) Int. Cl. A61B 17/88

(52) U.S. Cl. CPC A61B 17/8858

(57) ABSTRACT

The present invention relates to a medical expansion system (100) for the expansion of a bone (200), in particular for the expansion of a tubular bone, comprising an expansion unit (1) extending in a longitudinal direction (L) with a first end (EE1) and a second end (EE2), wherein the expansion direction of the expansion unit (1) is aligned optionally substantially radially (R); a connecting catheter (3) which is connected, with a first end (EV1) thereof, to a first end (EE1) of the expansion unit (1); and a port (5) which is connected with a second end (EV2) of the connecting catheter (3); wherein the expansion unit (1), the connecting catheter (3) and the port (5) are in fluid communication with each other and the expansion system (100) is fluid-tight to the outside.

The present invention relates to a medical expansion system for expanding a bone, to a kit including at least one medical expansion system for expanding a bone, and to a method for expanding a bone.

Bones are damaged in various ways in everyday life, for example on the one hand due to bone fractures caused by external influences (injuries) or by infections, or on the other hand due to tumors (osteosarcoma, Ewing's sarcoma). In some cases, healing of the damaged bone is no longer possible. One option is then bone transplantation, i.e. removal of a bone segment from another bone segment of the same organism that is not absolutely necessary for mobility. An example is the removal of a fibula segment (calf bone). The reduced functionality of the lower leg after a removal may be largely maintained by the tibia (shin bone). However, a fibula segment can only be transplanted directly to a limited extent if, for example, a femoral segment is to be replaced. After transplantation, the corresponding bone must be stabilized for a certain period of time using intramedullary nails or similar. However, the fibula segment is not suitable for such intramedullary nails due to its small diameter.

It is the object of the present invention to provide herein proposals for solving this problem.

The object of the present invention may be achieved by the medical expansion system having the features as disclosed herein, by the kit having the features as disclosed herein and by the method having the features as disclosed herein.

The medical expansion system, also shortly expansion system, serves in use for expanding a bone, in particular for expanding a tubular bone. It encompasses:
an expansion unit (e.g., at least one expansion balloon or dilatation balloon) extending in a longitudinal direction and having a first end and a second end. The expansion unit is preferably embodied to expand substantially radially, or more radially than longitudinally, e.g. at least 5 times or at least 10 times more radially than longitudinally.
a connecting catheter being connected with a first end thereof to a first end of the expansion unit; and
a port preferably being connected to, or being part of, a second end of the connecting catheter.

The expansion unit, the connecting catheter and the port, or their inner lumen, are in fluid communication with each other. The expansion system is fluid-tight towards the outside and/or accordingly self-contained, e.g. towards the environment.

The expansion system is preferably closed such that, over the application period of 5 to 80 days, there is no exchange of the fluid with the surrounding tissue or no leakage of the fluid present in the expansion system out of the expansion system into surrounding tissue, and no entry of surrounding tissue liquids into the expansion system.

The kit according to the present invention comprises at least one expansion system according to the present invention and at least one liquid, preferably a physiological saline solution (NaCl, e.g.), wherein the expansion system or the closed expansion unit is filled with this liquid during use. The method according to the present invention serves for expanding a bone, in particular for expanding a tubular bone. It comprises providing an expansion system or kit according to the present invention. It further comprises advancing the first end or section of the expansion unit via an access into an opened interior of the bone to implant the expansion unit wholly or partially between fragments or bone sections of the bone.

When it is disclosed herein that the subject-matter according to the present invention comprises one or several features in a certain embodiment, it is also respectively disclosed herein that the subject-matter according to the present invention does, in other embodiments, likewise according to the present invention, explicitly not comprise this or these features, for example, in the sense of a disclaimer. Therefore, for every embodiment mentioned herein it applies that the converse embodiment, e.g. formulated as negation, is also disclosed.

In all of the aforementioned and following statements, the use of the expression “may be” or “may have” and so on, is to be understood synonymously with “preferably is” or “preferably has”, and so on respectively, and is intended to illustrate embodiments according to the present invention.

Whenever numerical words are mentioned herein, the person skilled in the art shall recognize or understand them as indications of numerical lower limits. Hence, unless this leads to a contradiction evident for the person skilled in the art, the person skilled in the art shall comprehend for example “one” (or “a/an”) as encompassing “at least one”. This understanding is also equally encompassed by the present invention as the interpretation that a numerical word, for example, “one” (or “a/an”) may alternatively mean “exactly one”, wherever this is evidently technically possible in the view of the person skilled in the art. Both of these understandings are encompassed by the present invention and apply herein to all used numerical words.

Advantageous developments of the present invention are each subject-matter of the dependent claims and embodiments.

Whenever an embodiment is mentioned herein, it is to be understood as an exemplary embodiment according to the present invention which is not to be understood as limiting.

Embodiments according to the present invention may comprise one or several of the features mentioned supra and/or in the following in any combination unless the person skilled in the art considers such a combination to be technically impossible.

In several embodiments, the first end of the connecting catheter and the first end of the expansion unit are releasably connected to each other by a first connector.

In several embodiments, a second end of the connecting catheter and the port are releasably connected to each other by a second connector.

In several embodiments, the port comprises a membrane for puncturing the port in order to supply or to remove a fluid. This serves for varying the pressure prevailing in the expansion system and radial expansion of the expansion unit.

In several embodiments, the membrane comprises a membrane structure for being pierced by a cannula (e.g. injection needle).

The tightness of the expansion system with respect to the environment is maintained. Preferably, the membrane structure is embodied to ensure tightness during and after puncturing at a statically applied pressure of up to 20 bar.

In some embodiments, the expansion unit is, or comprises, a dilatation balloon.

In some embodiments, the expansion unit is a rotationally symmetric body.

In some embodiments, the expansion unit is an elongated body having a length that exceeds the diameter of the expansion unit by at least a factor of 2, 3, 5, or 10.

In several embodiments, the radial diameter of the expansion unit is at most 30 mm in the maximally expanded state.

In several embodiments, the radial diameter is at most 4 mm in an unexpanded state.

In several embodiments, the length of the expansion unit in its longitudinal alignment or direction is between 3 cm and 30 cm, for example, in a non-expanded state.

In several embodiments, the expansion system is a disposable or a single-use system, in others it is not.

In several embodiments, the expansion system comprises or consists of sterilizable and/or biocompatible materials and/or is sterilized.

In several embodiments, the components of the expansion system are pressure stable to at least 20 bar. In this way, a kink-resistant arrangement, in particular of the connecting catheter, may be effected. In addition, the required tightness of the system may be ensured.

In several embodiments, the expansion unit is provided within a surrounding optionally multilayer hose or tube, preferably an elastic plastic hose, and is expanded therein. Materials used for this are e.g. silicone, a thermoplastic elastomer, etc.

In several embodiments, the expansion unit is provided within, expanded within, and/or surrounded, at least in part, by a surrounding optionally multilayer mesh, fabric, or braid.

In several embodiments, the expansion unit has multiple, preferably different, diameters, cross-sections, and/or lumina along its longitudinal axis, with optionally several inflection points and/or different gradients.

In several embodiments, several expansion units, e.g. dilatation balloons, are combined in parallel or in series for expansion in the expansion unit, in the longitudinal and/or circumferential direction thereof.

In several embodiments, e.g. of the method according to the present invention, the expansion unit is expanded/deflated in a pressure controlled and/or volume-controlled manner, wherein the expansion is performed in at least 5 steps and the deflation may be performed in one or more steps.

In several embodiments, the components of the expansion system are provided and embodied to be connected in situ and/or, more particularly for the initial, filling with liquid in situ or emptying there.

In some embodiments, the liquid comprises pharmacological agents that can or are intended to ensure the shelf life, asepsis, sterility or other properties of the liquid over the implantation period, e.g. at least 30 days, 50 days, 80 days, or more.

In several embodiments, the components of the expansion system are pre-assembled or connected to each other at the factory. Thus, the expansion system may be implanted without any further assembly steps or connection steps.

In several embodiments, the expansion system comprises an elastic or non-elastic sheath, preferably manufactured or provided separately, for, at least partially, surrounding the, e.g. non-compliant or semi-compliant or compliantly manufactured expansion unit. The sheath may fully or partially surround the expansion unit and limit, mitigate, or the like, the extension and thus protect the expansion unit from damage.

In several embodiments, the sheath is embodied to allow a uniform, radial expansion and/or contraction of the expansion unit. This may be or may become effected or facilitated in particular by material properties and/or predetermined geometry.

In several embodiments, the connecting catheter is designed to be pressure-resistant and kink-resistant such that at pressures of up to 20 bar there is no, or no significant, that is e.g. not more than 5% circumferential increase, expansion of the connecting catheter to be observed, there occurs no kink in the connecting catheter at a minimum bending radius of 5 mm that interrupts fluid communication between the port and the expansion unit, and/or the connecting catheter preferably has a length of 2 cm to 50 cm. Such embodiments may advantageously allow the port to be placed in an anatomically preferred position.

In several embodiments, the radial diameter of the expansion unit is between 1 mm and 10 mm in a non-expanded state.

In several embodiments, the expansion system is embodied and provided to cause expansion of bone callus tissue and surrounding soft tissue during expansion of the expansion unit.

In several embodiments, some or all of the outer surfaces of the port and/or connecting catheter and/or expansion unit that may during use be in contact with the bone or surrounding tissue have a medication-based coating such as bone growth factor, antibiotics, antibacterial agents, etc.

In several embodiments, pharmacologically active agents, such as antibiotics, growth factors, etc., are integrated into the sheath, outer surfaces, or the like that are intended to be in tissue contact during use, the pharmacologically active agents preferably having a depot effect or long-term effect, and being delivered, e.g., over the application period of 15 to 80 days.

In several embodiments, the expansion system is embodied to remain fully implanted in the body over a period of 10 to 80 days. In some embodiments, e.g., of the method according to the present invention, it remains there that long.

In several embodiments, the expansion system does not comprise a connector (or it is not used) for an injection device for injecting liquid, rather the port, e.g. placed subcutaneously, is punctured using a cannula.

In some embodiments, the port is provided and embodied to be also implanted (and in some embodiments it is also implanted), e.g., subcutaneously.

In several embodiments, the expansion unit and/or the connecting catheter comprises a flexible or rigid support structure, e.g. a tube, core, sleeve, wire, rod, mesh, or any combinations thereof extending axially to the expansion unit.

In several embodiments, the support structure is stiffer or stronger than the innermost layer of the expansion unit.

In several embodiments, the support structure extends, at least in sections, through an interior of the expansion unit.

In several embodiments, the support structure extends through a central section of a cross-section and in longitudinal direction of the expansion unit.

In several embodiments, the port is or comprises a pump. It is embodied to convey or inject volumes into the expansion unit out of a volume reservoir at the instigation of a user or in an automated manner (such as by a control device that communicates wired or wirelessly with the pump).

In several embodiments, the expansion unit is embodied to be implanted in the bone after osteotomy between a free-moving bone fragment and the bone of origin.

In several embodiments, one to ten or more fixation eyelets and/or X-ray markers are attached to the expansion unit and/or connecting catheter to allow fixation of the expansion unit and/or connecting catheter via sutures to avoid dislocation during the period of use and/or enable position control by X-ray imaging.

In several embodiments, the kit further comprises an injection syringe, preferably with an injection needle for, preferably stepwise, filling or further filling the expansion system or the expansion unit with a liquid, preferably a physiological saline solution.

In several embodiments, the expansion system or the kit further comprises a first X-ray marker, preferably at the first end or region of the expansion unit, and/or a second X-ray marker, preferably at the second end or region of the expansion unit.

In several embodiments, the method further encompasses at least one of the steps:
opening the access to the interior, in particular to the medullary cavity, of the bone, preferably via the caput, e.g. the caput fibulae;
dilating the access by a dilatator, preferably via a guide wire;
opening a bone window on the shaft of the bone by osteotomy; and/or
connecting the interior with a protective sleeve.

In several embodiments, the method further comprises gradually expanding the expansion unit by volume injection through the port after a latency period of about 3 to 12 days during which bone callus tissue has formed between osteotomized bone of origin and loosened bone fragment, thus resulting in callus distraction of the bone callus tissue. This may be effected by a control device of the pump or a syringe pump, or manually by the user.

In several embodiments, one, several or all of the parts or sections of the X-ray marker consist of or comprise a cobalt-chromium (CoCr) alloy.

In some embodiments, one, several or all of the parts or sections of the X-ray marker consist of titanium, particularly grade 5 titanium, or comprise titanium.

In several embodiments, one, several or all of the parts or sections of the X-ray marker consist of wholly or partially of pyrocarbon and/or ceramic.

In some embodiments, one, several or all of the parts or sections of the X-ray marker consist of or comprise, partially or in whole, a plastic (in particular polyethylene, in particular highly cross-linked polyethylene, in particular with vitamin E).

In several embodiments, the expansion system is fully implantable, or is already fully implanted.

In some embodiments, the expansion system does not have a pressure source or liquid source, the expansion unit is not in fluid communication therewith.

In several embodiments, the expansion system and/or the kit does not comprise a control device by which an actuator, such as a pump, would be controlled, in particular according to a predetermined algorithm or, in particular automatic or programmed, course, to act on the expansion unit, e.g. by liquid.

In several embodiments, some or all of the advantages mentioned supra or below are achievable.

The expansion system according to the present invention may be temporarily implanted completely due to its optional outwardly fluid-tight structure. Thus, advantageously, a risk of infection may be reduced or completely avoided. This has a very high clinical importance, since infections in the bone sections may have serious consequences.

Further advantages may be the vitality of the expanded bone obtained according to the invention, the fact that 100% autologous material can be provided to repair the bone defect, that a completed procedure is possible when expanding the “donor” bone and the standardized method for this.

The high biomechanical quality, the rapid healing associated with early mobilization and the low surgical risk may also be advantageous.

In the following, the present invention, in particular the medical expansion system, is described based on preferred embodiments. The present invention is nevertheless not limited to these embodiments. The following applies in the figures:

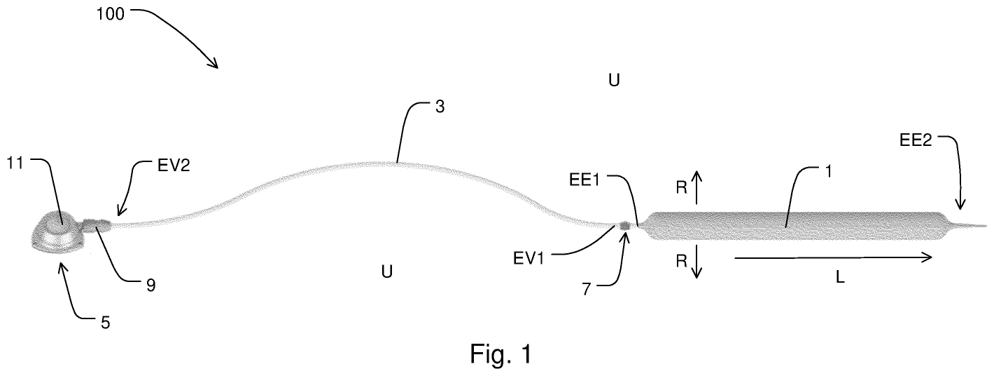

FIG. 1 shows the medical expansion system according to the present invention of an embodiment in perspective view;

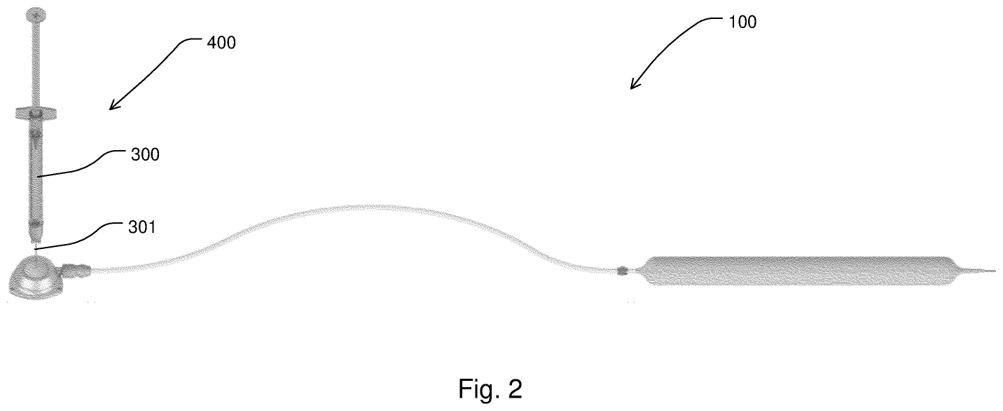

FIG. 2 shows the kit according to the present invention of an embodiment in perspective view;

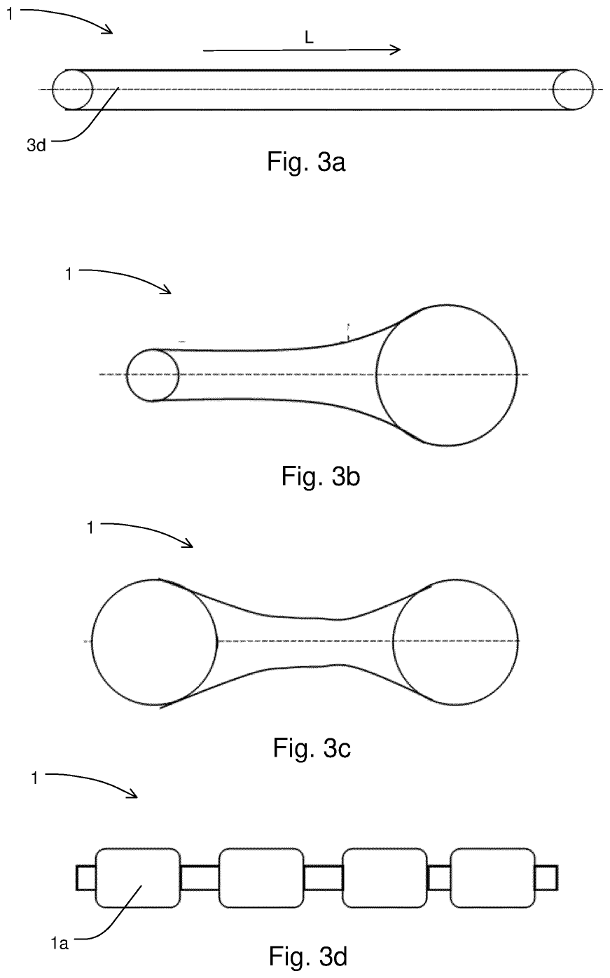

FIG. 3a-c show the expansion unit in various embodiments;

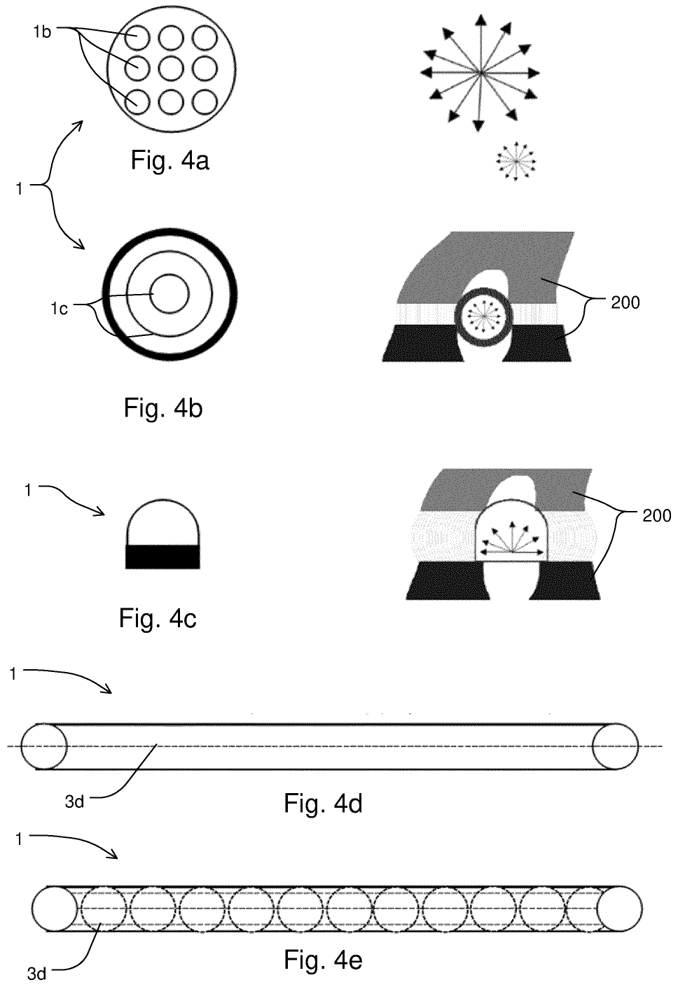

FIG. 4a-e show further embodiments of the expansion unit;

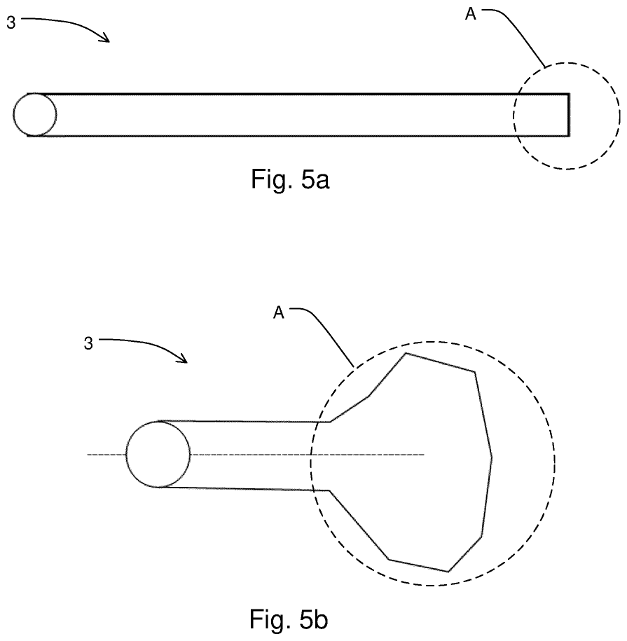

FIG. 5a,b show the connecting catheter in various embodiments;

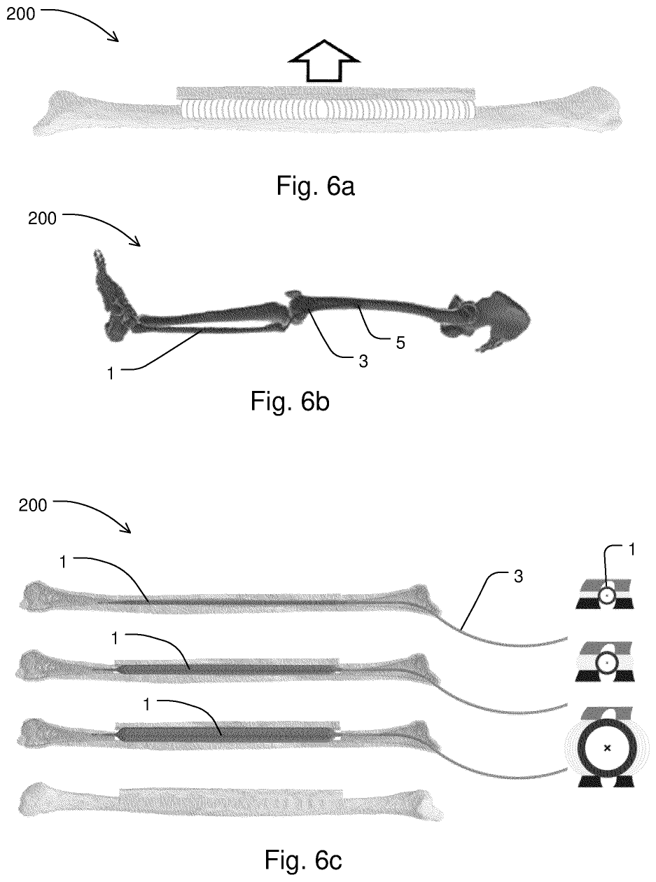

FIG. 6a -c show a schematic course for applying the medical expansion system according to the present invention to a tubular;

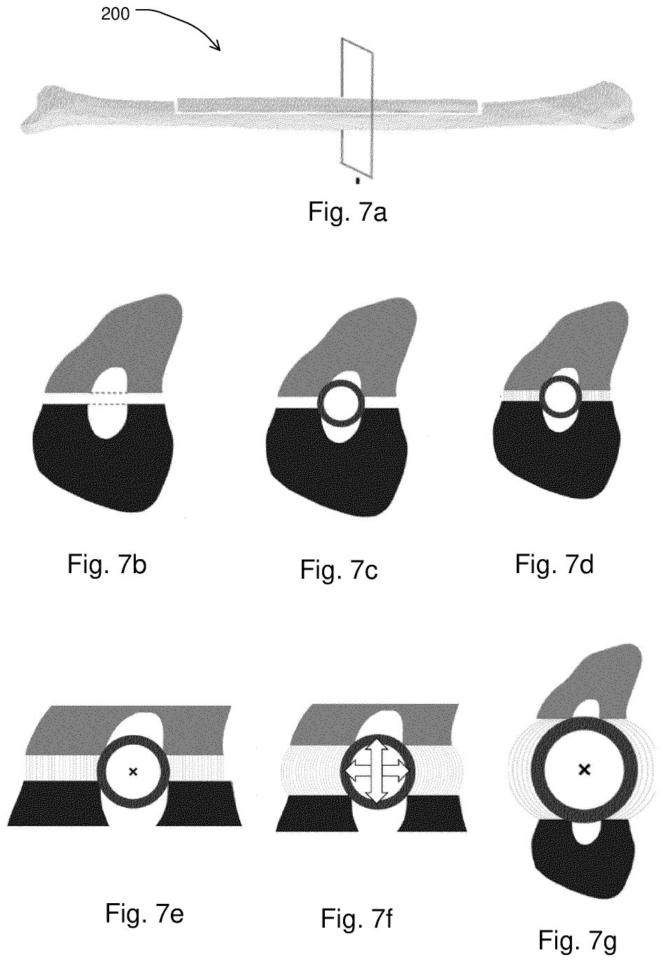

FIG. 7a -g show schematically simplified the individual steps or phases of expansion of the tubular bone by the expansion system according to the present invention;

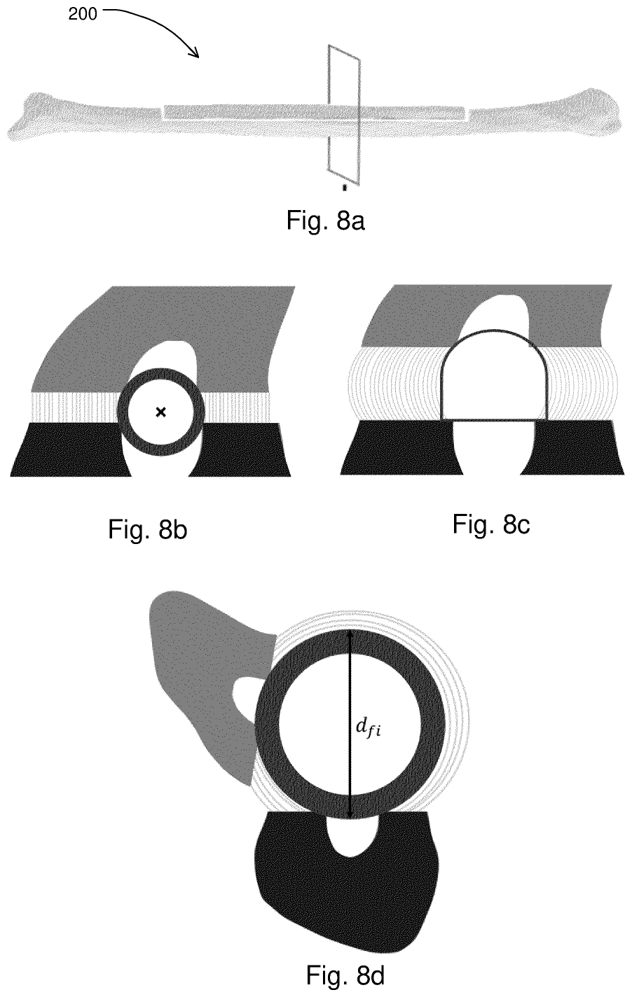

FIG. 8a -d show further details of the individual steps of FIG. 7;

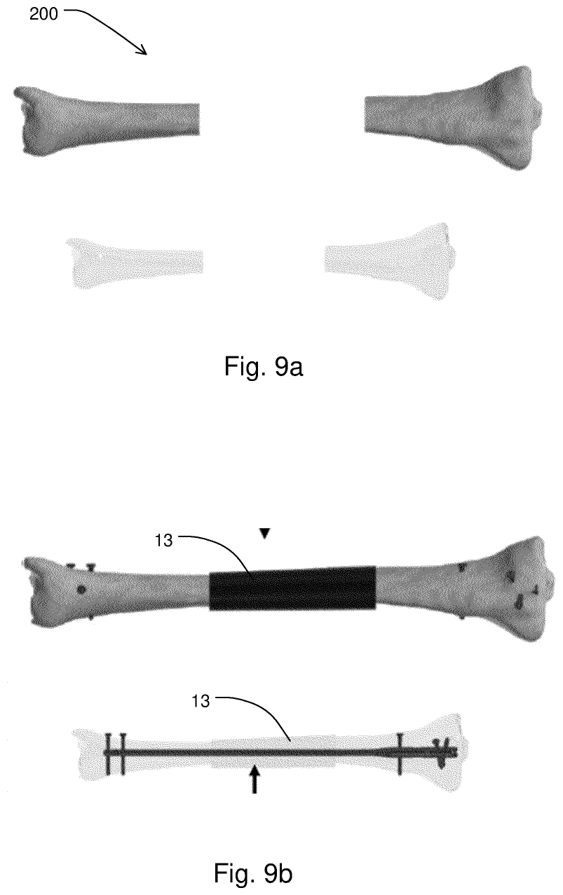

FIG. 9a,b show the removal of a bone segment and the preparation of the bone for bone transplantation using the expansion system according to the present invention;

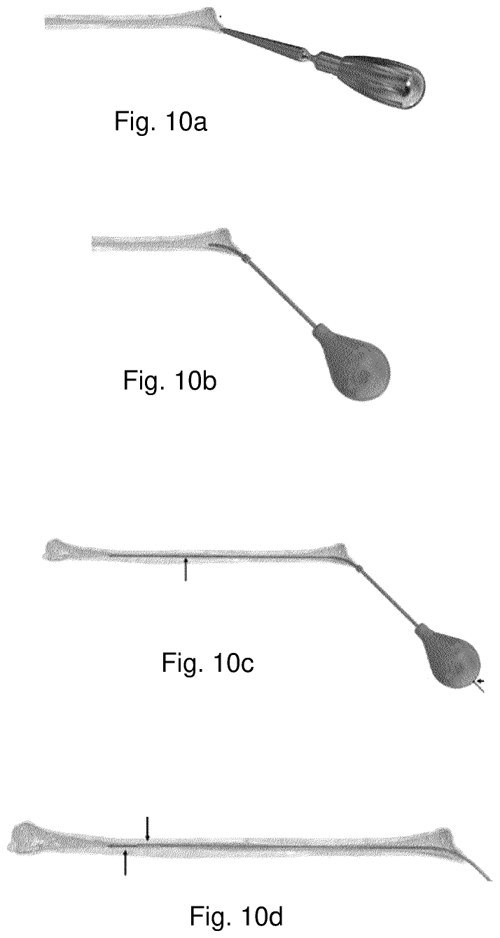

FIG. 10a-d show the steps following the steps of FIG. 9 for preparing the bone to be transplanted using the expansion system according to the present invention;

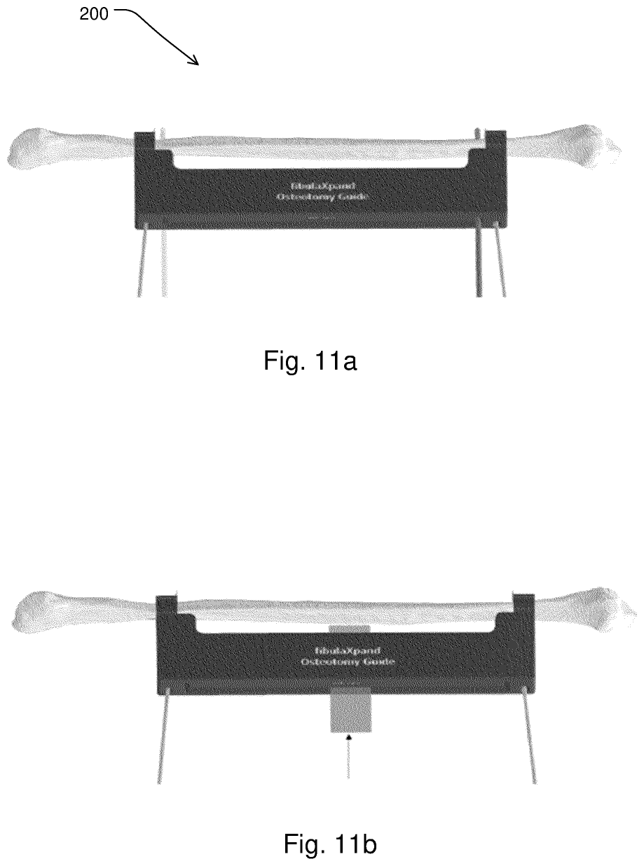

FIG. 11a,b show the steps of an osteotomy following the steps of FIG. 10 for preparing the bone to be transplanted using the expansion system according to the present invention;

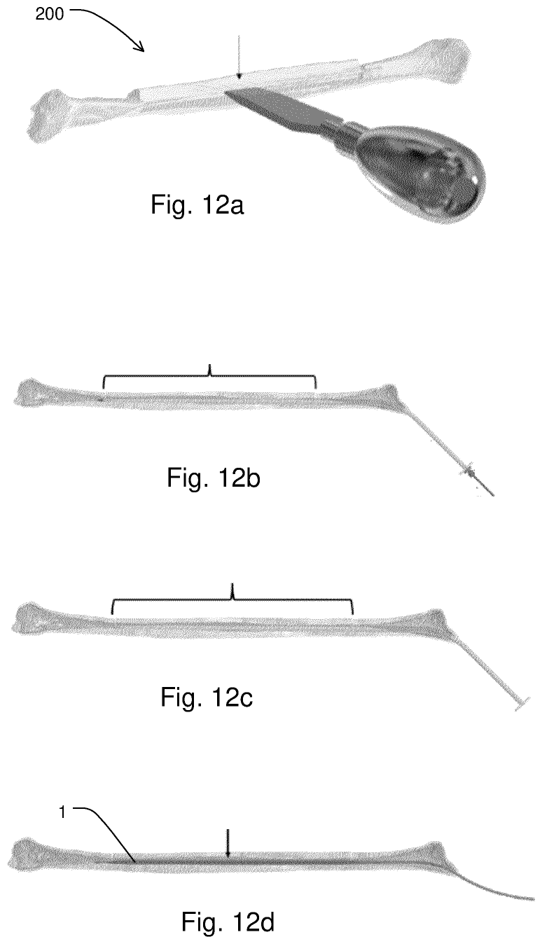

FIG. 12a-d show further steps possible for preparing the bone to be transplanted and the use of the expansion system according to the present invention; and

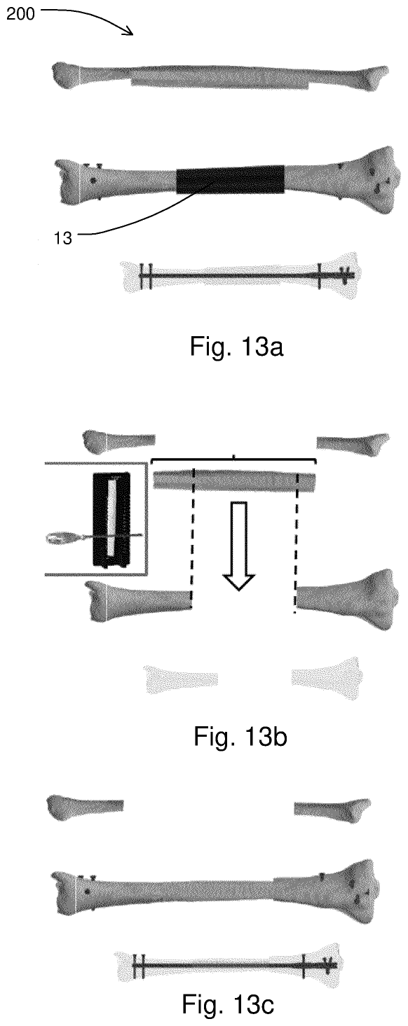

FIG. 13a-c show the final bone transplantation of the transplant expanded with the aid of the expansion system according to the present invention.

FIG. 1 shows an exemplary embodiment of the medical expansion system 100 according to the present invention, partly from the side and partly in perspective view.

The medical expansion system 100, referred to herein and also hereinafter shortly as expansion system 100, comprises an expansion unit 1 extending in a longitudinal direction L and having a first end EE1 and a second end EE2. The expansion direction of the expansion unit 1 is aligned or directed substantially in the radial direction R.

Further, the expansion system 100 comprises a connecting catheter 3 connected by or with a first end EV1 thereof to the first end EE1 of the expansion unit 1. Die Verbindung kann mittels eines optionalen, ersten Konnektors 7 erfolgen, sie kann alternativ stoffschltissig erfolgen. The connection may be established by an optional, first connector 7, it may alternatively be established in a material-fit manner.

The expansion system 100 further comprises a port 5 connected to a second end EV2 of the connecting catheter 3. The connection may be established by an optional, second connector 9, it may alternatively be established in a material-fit manner.

The port 5 is provided here exemplarily with a membrane 11. It fluidically seals the fluid system of the expansion system 100 from the outside. In the embodiment shown here, the membrane 11 may be provided in order to be punctured, e.g. by a cannula 301, see FIG. 2. Fluid may be supplied to the sealed fluid system via the cannula 301 by a syringe 300 as also shown in FIG. 2.

FIG. 2 shows the kit 400 according to the present invention of an embodiment in perspective view.

The kit 400 optionally comprises in this embodiment an expansion system 100 according to the present invention, which is filled with a liquid, preferably a physiological saline solution, and a syringe 300 with a cannula 301 for, preferably stepwise, further filling of the expansion system 100 with liquid from the syringe 300.

The expansion unit 1, the connecting catheter 3 and the port 5 are in fluid communication with each other. The expansion system 100 is fluid-tight to the outside towards the environment U. In other words, the expansion system 100 is self-contained.

FIGS. 3a-c show the expansion unit 1 in various embodiments.

FIG. 3a shows the expansion unit 1 in an optional tubular embodiment, in which the outer diameter is constant over the length L. An optional internal concentric support structure 3d may advantageously increase the stability of the expansion unit 1 before and/or during implantation. Purely exemplarily, the concentric support structure 3d may be a wire, a rope, a rod, a fabric or similar.

FIG. 3b shows the expansion unit 1 in an optional embodiment widening in a trumpet shape towards an end section. This may be used advantageously, to particularly strongly expand or widen for example certain longitudinal sections of a tubular bone.

FIG. 3c shows the expansion unit 1 in an optional embodiment that expands in a trumpet shape toward both end sections.

FIG. 3d shows the expansion unit 1 in a further, optional embodiment. The widening or expansion optionally takes place in individual sections 1a or segments 1a along the entire longitudinal extent of the expansion unit 1. The segments 1a may be of uniform or different design, for example of different lengths, with different diameters, with different materials and/or of different structure. This may advantageously be applied for different bone structures and/or implantation sections with regard to the surrounding soft tissue structures and/or the course of the outer contour of the bone.

FIGS. 4a-e show further embodiments of the expansion unit 1.

FIG. 4a shows an optional expansion unit 1 in a multi-lumen embodiment. The lumina 1b arranged in parallel may be arranged uniformly or differently within an outer sheath (shell, jacket). Thus, the expansion of the bone may advantageously be done selectively in order to affect different bone sections and/or surrounding tissue structures differently. The illustration to the right of FIG. 4a shows in a schematically simplified manner how the expansion pressure of the individual lumina may affect the expansion unit 1.

FIG. 4b shows a further, optional expansion unit 1 in a multi-lumen embodiment, wherein the individual lumina 1c are arranged concentrically and/or in multiple layers. In the illustration to the right of FIG. 4b, the expansion of an osteotomized bone 200 is shown schematically simplified. A callus may form between the bone segments, in particular stepwise.

FIG. 4c shows an optional expansion unit 1 in a further embodiment, wherein the expansion here does not occur uniformly radially over the circumference. The selective radial expansion over the circumference is achieved by different structures of the expansion unit 1, as shown exemplarily in FIG. 4c. The corresponding selective expansion of the bone 200 is shown schematically simplified in the illustration to the right of FIG. 4c.

FIG. 4d shows the expansion unit 1 in a further optional embodiment, which corresponds essentially to the illustration of FIG. 3a. The optional core supporting the expansion unit 1, which itself does not expand, may be of rigid or flexible design.

FIG. 4e shows the expansion unit 1 in a further optional embodiment, wherein the supporting structures 3d are optionally additionally arranged in sections outside the core. The support structure 3d may comprise or consist of a fabric or mesh. The support structure 3d may be arranged concentrically around a core.

FIG. 5a,b show the connecting catheter 3 in various embodiments.

FIG. 5b shows the connecting catheter 3 with an optional end section A that renders possible a direct introduction of a liquid into the expansion system 100. Similar to an introduction using a separate port 5, the end section A may include for example a membrane 11 for being punctured by an injection needle 301. In this way, the connecting catheter 3 requires no connection to a port, or its end section A with the membrane 11 is the port.

FIG. 5a shows the connecting catheter 3 with a further, optional end section A, which may have for example a cylindrical, oval, rectangular or other shape.

FIGS. 6a-c show a schematic course for applying the medical expansion system 100 according to the present invention to a tubular bone 200 or an embodiment of the method according to the present invention.

FIG. 6a shows schematically simplified an expansion of a bone segment osteotomized on the longitudinal side. The direction of expansion is indicated by the arrow shown radially, perpendicular to the longitudinal direction. By a gradual expansion, callus may form between the bone segments which gradually move away from each other. This is shown by the individual, arc-shaped lines.

FIG. 6b shows schematically simplified an optional arrangement of the expansion system 100 in the section of the lower extremities of a human being. The expansion unit 1 is arranged in the fibula (calf bone) directly adjacent to the tibia (shin bone). The connecting catheter 3 connects the expansion unit 1 to a port 5, which is arranged in the section of the femur (thigh bone).

FIG. 6c shows schematically simplified, in the four individual illustrations from top to bottom, with the respective cross sections on the right, the stepwise expansion of the osteotomized tubular bone 200 by the expansion system 100 according to the present invention over time (from top to bottom).

FIG. 7a-g show schematically simplified the individual steps for expansion of the osteotomized tubular bone 200 using the expansion system 100 according to the present invention.

FIG. 8a-d show further details of the individual steps of FIGS. 7a-g.

FIG. 9a,b show the removal of a bone segment to be replaced by a transplanted bone transplant being prepared using the expansion system 100 according to the present invention. Until the bone transplant is sufficiently expanded by the expansion system 100, the section of the removed bone segment is temporarily aided or provided with a so-called spacer 13.

FIG. 10a-d show the steps following FIG. 9 for preparing the bone segment to be transplanted with the aid of the expansion system 100 according to the present invention.

FIG. 11a,b show the steps of an osteotomy following FIG. 10 for preparing the bone to be transplanted with the aid of the expansion system 100 according to the present invention.

FIG. 12a-d show the further steps to be performed in order to prepare the bone 200 to be transplanted and the insertion of the expansion system 100 according to the present invention.

FIG. 13a-c show the final bone transplantation of the transplant being expanded with the aid of the expansion system 100 according to the present invention.

The pages of figures following FIGS. 1 to 13c again show the subject-matters according to the present invention in exemplary embodiments. The subject-matters shown therein may by the present invention be combined with any other features disclosed herein in any combination.

### LIST OF REFERENCE NUMERALS

100 medical expansion system
200 bones
300 syringe
301 cannula
400 kit
EE1 first end of the expansion unit
EE2 second end of the expansion unit
EV1 first end of the connecting catheter
EV2 second end of the connecting catheter
L longitudinal direction
R radial
U environment
1 expansion unit
1a section, segment of the expansion unit
1b parallel lumen of the expansion unit
1c concentric lumen of the expansion unit
3 connecting catheter
3d support structure
5 port
7 first connector
9 second connector
11 membrane
13 spacer

1. A medical expansion system for expanding a tubular bone, comprising
an expansion unit extending in a longitudinal direction with a first end and a second end, wherein the expansion direction of the expansion unit (1) is aligned substantially radially;
a connecting catheter connected with a first end thereof to a first end of the expansion unit; and
a port connected to a second end of the connecting catheter;
wherein the expansion unit, the connecting catheter and the port are in fluid communication with each other and the medical expansion system is fluid-tight to the outside.

2. The medical expansion system according to claim 1, wherein the first end of the connection catheter and the first end of the expansion unit are releasably connected to each other by a first connector.

3. The medical expansion system according to claim 1, wherein a second end of the connection catheter and the port are releasably connected to each other by a second connector.

4. The medical expansion system according to claim 1, wherein the port comprises a membrane for puncturing the port for supplying or withdrawing a fluid.

5. The medical expansion system according to claim 4, wherein the membrane comprises a membrane structure for being pierced with a cannula.

6. The medical expansion system according to claim 1, wherein the expansion unit is, or comprises, a dilatation balloon.

7-11. (canceled)

12. The medical expansion system according to claim 1, wherein the expansion unit expands within a surrounding hose, tube, mesh, fabric or braid.

13. (canceled)

14. The medical expansion system according to claim 1, wherein the expansion unit comprises along its longitudinal axis several different diameters, cross-sections and/or lumina.

15. The medical expansion system according to claim 1, wherein the expansion unit combines several expansion units or dilatation balloons in parallel or in series for the expansion in the longitudinal or circumferential direction.

16-18. (canceled)

19. The medical expansion system according to claim 1, comprising an elastic sheath for at least partially surrounding the non-compliant or semi-compliant or compliantly manufactured expansion unit.

20-26. (canceled)

27. The medical expansion system according to claim 1, wherein for injecting liquid there is no connection made via a connector of an injection device, but the subcutaneously placed port is punctured with a cannula.

28. The medical expansion system according to claim 1, wherein the expansion unit and/or the connecting catheter comprises an axial, flexible/rigid support structure.

29. The medical expansion system according to claim 1, wherein the port is or comprises a pump, embodied to convey or inject volume into the expansion unit out of a volume reservoir at the instigation of a user or in an automated manner.

30. (canceled)

31. The medical expansion system according to claim 1, wherein one to ten or more fixation eyelets and/or X-ray markers are attached to the expansion unit and/or to the connecting catheter, hence enabling the fixation of the expansion unit and/or of the connecting catheter via sutures in order to avoid dislocation during the period of use and/or enable position control by X-ray imaging.

32. A kit comprising
at least one medical expansion system according to claim 1; and
at least one liquid, wherein the medical expansion system or the closed expansion unit is filled with said liquid.

33. The kit according to claim 32, further comprising
an injection syringe, preferably with an injection needle for filling or further filling the medical expansion system or the expansion unit with a liquid.

34. The kit according to claim 32, further comprising
a first X-ray marker at the first end or section of the expansion unit, and/or a second X-ray marker at the second end or region of the expansion unit.

35. A method for expanding a tubular bone, encompassing the steps of:
providing an medical expansion system according to claim 1;
advancing the first end or section of the expansion unit via an access into an opened interior of the bone.

36. The method according to claim 35, further encompassing at least one of the steps:
opening the access to the interior of the bone;
dilating the access using a dilatator;
opening a bone window at the shaft of the bone by osteotomy; and/or
connecting the interior with a protective sleeve.

37. The method according to claim 35, wherein stepwise expansion of the expansion unit by volume injection via port takes place after a latency phase of about 3 to 12 days during which bone callus tissue has formed between osteotomized bone of origin and loosened bone fragment, thus leading to callus distraction of the bone callus tissue.
